(19) United States
(12) Patent Application Publication
(10) Pub. No.: US 2013/0012951 A1
(43) Pub. Date: Jan. 10, 2013

(54) SYSTEMS AND METHODS FOR TREATING A SPINE THROUGH A SINGLE VERTEBRAL BODY INSERTION POINT

(75) Inventor: Evan D. Linderman, Northbrook, IL (US)

(73) Assignee: CAREFUSION 207, INC., San Diego, CA (US)

(21) Appl. No.: 13/178,958

(22) Filed: Jul. 8, 2011

(51) Int. Cl. A61B 17/58

(52) U.S. Cl. 606/93

(57) ABSTRACT

Methods for treating a spine include inserting a distal end of a cannula into a vertebral body. A distal segment of an access needle is inserted into the cannula lumen. The distal segment terminates at a distal tip and has a shape memory characteristic naturally assuming a curved shape in longitudinal extension. The cannula forces the distal section to deflect from the curved shape toward a straightened shape. The distal segment is distally advanced into bone structure of the vertebral body and naturally reverts toward the curved shape. With further distal advancement, the distal tip progresses through an end plate of the vertebral body. Finally, a structure of the spine is altered in at least one of: delivering a curable material, creating a cavity, or aspirating nucleus material.

### BACKGROUND

The present disclosure relates to methods and systems for treating a spine of a patient. More particularly, it relates to methods and systems for accessing various target sites of the spine through a single vertebral body insertion point, for example in delivering a stabilizing material.

Surgical intervention at damaged or compromised bone sites has proven highly beneficial for patients, for example patients with back pain associated with vertebral damage.

Bones of the human skeletal system include mineralized tissue that can be generally categorized into two morphological groups: “cortical” bone and “cancellous” bone. Outer walls of all bones are composed of cortical bone, which has a dense, compact bone structure characterized by a microscopic porosity. Cancellous or “trabecular” bone forms the interior structure of bones. Cancellous bone is composed of a lattice of interconnected slender rods and plates known by the term “trabeculae”.

During certain bone-related procedures, cancellous bone is supplemented by an injection of a palliative (or curative) material employed to stabilize the trabeculae. For example, superior and inferior vertebrae in the spine can be beneficially stabilized by the injection of an appropriate, curable material (e.g., PMMA or other bone cement or curable material). In other procedures, percutaneous injection of stabilization material into vertebral compression fractures by, for example, transpedicular or parapedicular approaches, has proven beneficial in relieving pain and stabilizing damaged bone sites. Such techniques are commonly referred to as “vertebroplasty”. Other skeletal bones (e.g., the femur) can be treated in a similar fashion. Regardless, bone in general, and cancellous bone in particular, can be strengthened and stabilized by palliative insertion or injection of bone-compatible material.

A conventional vertebroplasty technique for delivering the bone stabilizing material entails placing an access cannula with an internal stylet into the targeted vertebral body delivery site. The access cannula and stylet are used in combination to pierce the cutaneous layers above the hard tissue to be supplemented, then to penetrate the hard cortical bone of the vertebral body, and finally to traverse into the softer cancellous bone underlying the cortical bone. Once positioned in the cancellous bone, the stylet is removed, leaving the access cannula in an appropriate, lodged position for delivery of curable material (e.g., via a needle or tube inserted through the access cannula) to the trabecular space of the vertebral body that in turn reinforces and solidifies the target site. In related procedures, a balloon or other expandable device is employed to form a cavity or void within the cancellous bone, with the curable material being then deposited into the cavity.

In some instances, a patient has multiple vertebral bodies requiring vertebroplasty treatment (e.g., two or more fractured vertebral bodies). Under these circumstances, current vertebroplasty methods entail multiple needle punctures in the patient. For example, if a patient has vertebral body fractures at levels L1 and L2, the clinician must separately lodge access cannulas in both the L1 and L2 vertebral bodies. These multiple percutaneous insertion points give rise to increased risks to the patient, time for the surgical procedure, and cost.

In light of the above, a need exists for improved methods and systems for accessing and treating multiple vertebral bodies of a patient, and other procedures entailing percutaneous access to a segment of the spine.

### SUMMARY

Some aspects in accordance with principles of the present disclosure relate to a method for treating a spine of a patient. The spine includes a first vertebral body connected to a second, immediately adjacent vertebral body by an intervertebral disc. The intervertebral disc, in turn, includes an annulus and a nucleus, and is bounded by a first end plate and a second end plate. The first vertebral body forms the first end plate, and the second vertebral body forms the second end plate. The method includes lodging a distal end of a guide cannula or needle into the first vertebral body. The guide cannula defines a linear lumen. A distal segment of an access needle is inserted into the guide cannula lumen. The distal segment terminates at a distal tip and has a shape memory characteristic naturally assuming a curved shape in longitudinal extension. With insertion of the distal section into the lumen, the cannula forces the distal section to deflect from the curved shape toward a straightened shape. The needle distal tip is then distally advanced from the distal end of the cannula and into bone structure of the first vertebral body. In this regard, at least a portion of the distal segment now distal the cannula distal end naturally self-reverts toward the curved shape. With further distal advancement of the access needle relative to the guide cannula, the distal tip progresses through the bone structure and then the first end plate of the vertebral body, forming a curved channel in the bone structure. Finally, a structure of the spine is altered in at least one of three manners. A curable material is delivered into the second vertebral body through the access needle. In addition or alternatively, a cavity forming device is delivered through the channel created by the access needle, and operated to form a cavity in the second vertebral body. Alternatively or in addition, a portion of the nucleus is removed from the patient through the access needle. In some embodiments, the method further includes accessing, and delivering curable material into, vertebral bodies immediately inferior and superior the first vertebral body.

Other aspects of the present disclosure relate to a kit for treating a spine of a patient via a single vertebral body insertion point. The kit includes a guide cannula and a plurality of access needles. The guide cannula defines a linear lumen and is adapted for percutaneous insertion into a vertebral body. Each of the access needles are sized to be slidably received within the cannula lumen, and each has a distal segment terminating at a distal tip. Further, each of the distal segments has a shape memory characteristic naturally assuming a curved shape in longitudinal extension, is deflectable to a more straightened shape when inserted within the cannula, and self-reverts back toward the memory set curved shape when removed from the cannula. With this in mind, the distal segment of a first one of the access needles differs from the distal segment of a second one of the access needles in terms of at least one of length and radius of curvature. In some embodiments, the kit further includes a template that visually represents the difference(s) between distal segments of the first and second access needles. With this configuration, the kit can be employed to perform a desired spinal procedure by initially lodging a distal end of the guide cannula within the vertebral body, and then comparing the template with the achieved cannula distal end location to facilitate selection of a “best fit” access needle from the available needles for subsequent deployment through the cannula.

### BRIEF DESCRIPTION OF THE DRAWINGS

FIG. 1 is an exploded view of a system for treating a spine of a patient in accordance with principles of the present disclosure;

FIG. 2A is a side view of a human spine;

FIG. 2B is a simplified superior view of a vertebrae of the spine of FIG. 2A;

FIG. 2C is a simplified lateral view of the vertebra of FIG. 2B;

FIG. 2D is a simplified cross-sectional view of an intervertebral disc of the spine of FIG. 2A;

FIG. 3 is a perspective view of a spinal segment for treatment by the system of FIG. 1, including a bone vertebra, superior and inferior vertebrae, and superior and inferior discs

FIGS. 4A and 4B illustrate initial lodging of a cannula component of the system of FIG. 1 within the base vertebral body in accordance with methods of the present disclosure;

FIGS. 5A-5D illustrate use of the system of FIG. 1 in treating the spinal segment of FIG. 3 to deliver a curable material into the superior vertebral body;

FIGS. 6A-6D illustrate another method in accordance with principles of the present disclosure performed on the spinal segment;

FIGS. 7A and 7B illustrate another method in accordance with principle of the present disclosure performed on the spinal segment;

FIGS. 8A-8D illustrate another method in accordance with principles of the present disclosure performed on the spinal segment, including delivering a curable material to the inferior vertebral body;

FIGS. 9A and 9B illustrate another method in accordance with principles of the present disclosure performed on the spinal segment, including delivering a curable material to the base vertebral body;

FIGS. 10A and 10B illustrate another method in accordance with principles of the present disclosure performed on the spinal segment, including aspirating nucleus material;

FIGS. 11A and 11B illustrate use of the system of FIG. 1 in locating an access cannula relative to a vertebral body target site;

FIG. 12 is an exploded view of a kit in accordance with principles of the present disclosure; and

FIGS. 13A and 13B illustrate use of the kit of FIG. 12 in treating a spine of a patient.

### DETAILED DESCRIPTION

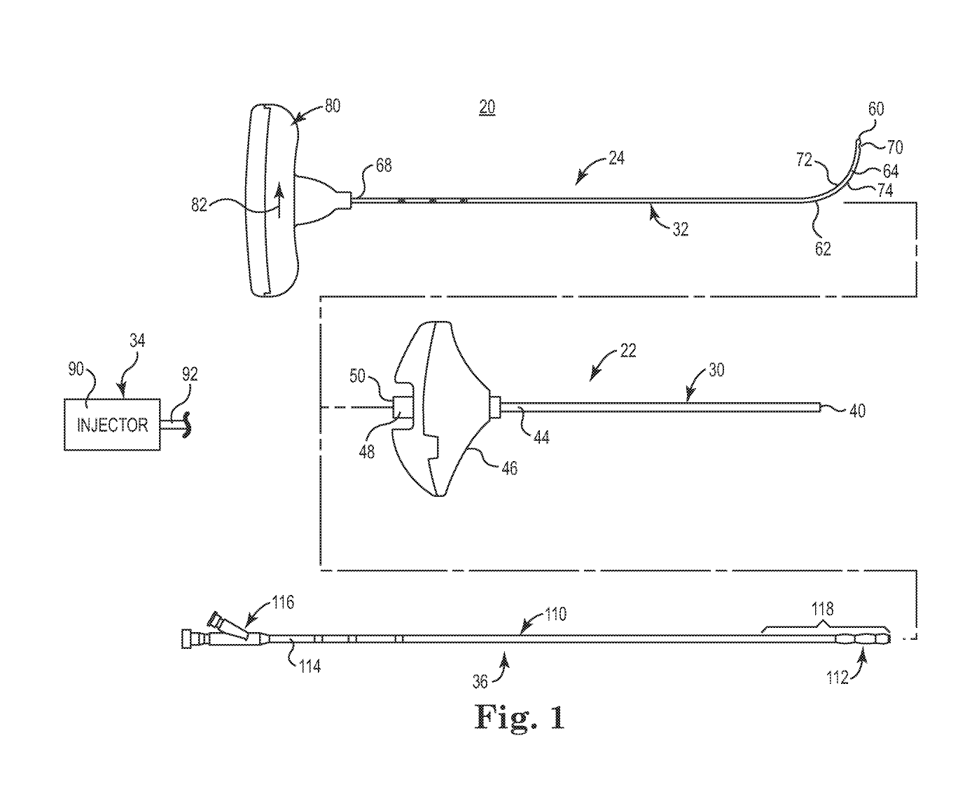

One embodiment of a system 20 for treating a spine of a patient in accordance with principles of the present disclosure is shown in FIG. 1. The system includes a cannula assembly 22 and an access needle assembly 24. Details on the various components are provided below. In general terms, however, the cannula assembly 22 includes a guide cannula 30 for percutaneous insertion into a vertebral body. The access needle assembly 24 includes an access needle 32. Once the guide cannula 30 is desirably located relative to the vertebral body (e.g., pedicular approach), a portion of the access needle 32 is delivered to the vertebral body via the cannula 30 and extended distally therefrom to form a curved channel toward and into an anatomical structure of interest adjacent the vertebral body (e.g., an adjacent intervertebral disc, an inferior or superior vertebral body, etc.). Once the access needle 32 is desirably located, further procedures are formed on the so-accessed anatomical structure. For example, in some embodiments, the system 20 includes an optional material delivery device 34 that is operated to deliver a curable material into the accessed anatomical structure via the access needle 32. Alternatively or in addition, the system 20 includes an optional cavity forming device 36 that is operated to form a cavity along the curved channel. In yet other embodiments, the system 20 is operable to remove material from the accessed anatomical structure (e.g., nucleus material of an intervertebral disc) via the access needle 32. Regardless, the system 20 and related methods of use facilitate treatment of various regions of a patient's spine via a single insertion point. For example, with the systems and methods of the present disclosure, vertebroplasty can be performed on multiple vertebral bodies with only a single needle puncture in the patient.

The system 20 and related methods of the present disclosure can be used for a number of different spine-related procedures, and are highly useful for delivering a curable material in the form of a bone curable material. The phrase “curable material” within the context of the substance that can be delivered by the system 20 and methods of the present disclosure is intended to refer to materials (e.g., composites, polymers, and the like) that have a fluid or flowable state or phase and a hardened, solid or cured state or phase. Curable materials include, but are not limited to, injectable bone cements (such as polymethylmethylacrylate (PMMA) bone curable material), which have a flowable state wherein they can be delivered (e.g., injected) by a cannula or needle to a site, and subsequently cured to hardened, cured material. Other materials such as calcium phosphates, bone ingrowth materials, antibiotics, proteins, etc., can be used in place of, or to augment, bone cement (but do not affect an overriding characteristic of the resultant formulation having a flowable state and a hardened, solid, or cured state). This would allow the body to reabsorb the curable material and/or improve the clinical outcome based on the type of filler implant material.

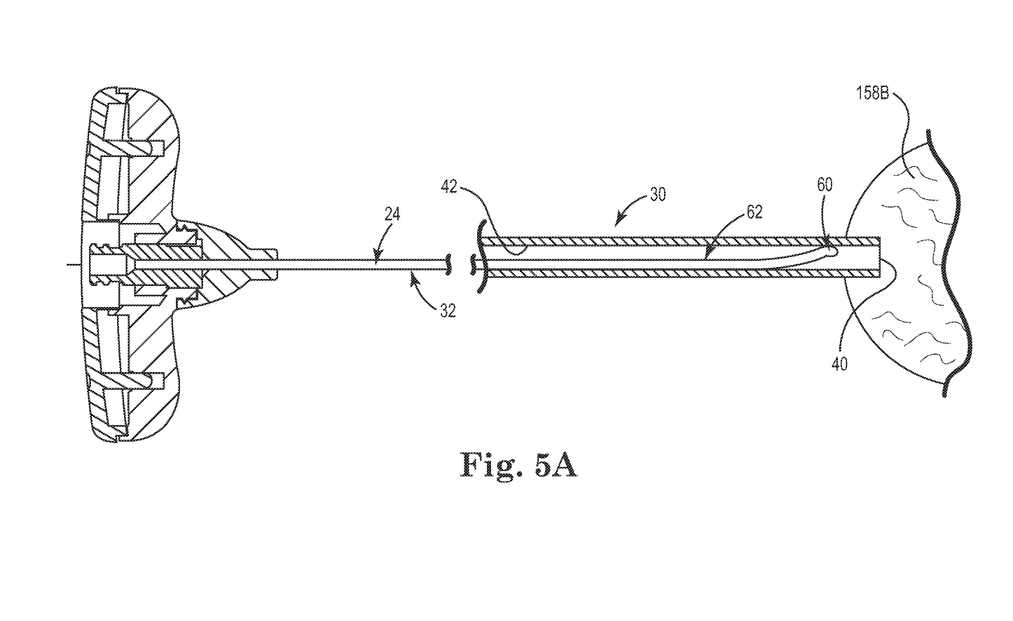

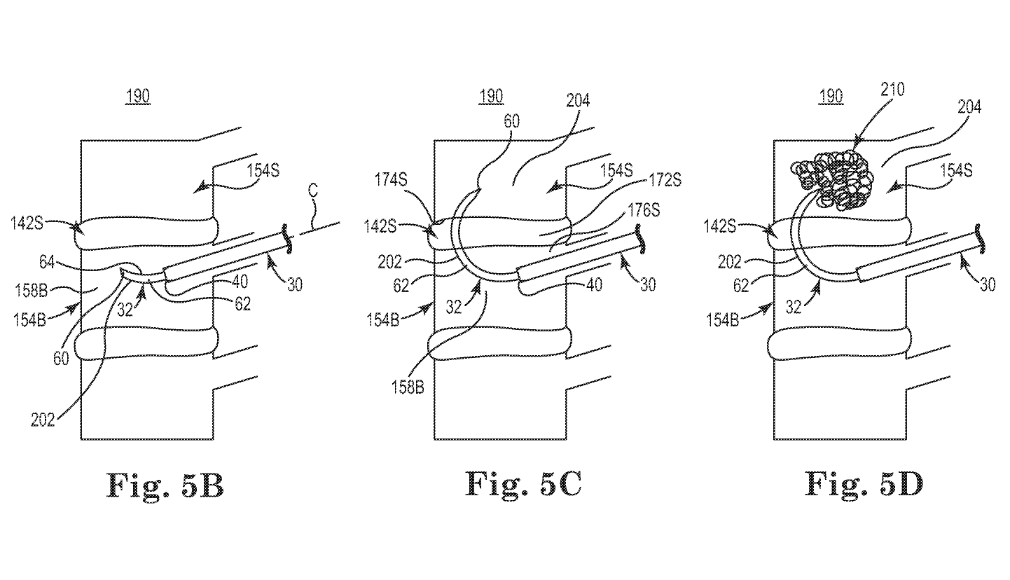

As mentioned above, the cannula assembly 22 includes the guide cannula 30. The guide cannula 30 terminates at a distal end 40, and defines a lumen 42 (hidden in FIG. 1 (shown in FIG. 5A)) extending from the distal end 40 to a proximal portion 44. The cannula 30 is akin to a needle and has a rigid construction. The cannula 30 can be made of a surgical grade of stainless steel, but may instead be made of known equivalent materials that are both biocompatible and substantially non-compliant at expected operated pressures. The lumen 42 defined by the cannula 30 is relatively linear (e.g., within 5° of a truly linear arrangement) and is sized to allow various equipment, such as the access needle 32, to pass therethrough. In some constructions, the distal end 40 is relatively blunt, but can alternatively be beveled to ease penetration of the cannula 30 through the cutaneous and soft tissues, and especially through hard tissues.

Surrounding the proximal portion 44 of the guide cannula 30 is an optional handle 46. In some construction, the cannula assembly 22 further includes a handle connector 48. The handle connector 48 is fluidly connected to the lumen 42, and defines a proximal end 50 of the cannula 30. Alternatively, the handle connector 48 can incorporate features forming part of a locking mechanism of the system 20. For example, the handle connector 48 can optionally include a luer-lock type of connector, but other known connecting mechanisms may be successfully interchanged (e.g., a conventional threaded hole, a threaded locking nut arrangement, etc.). Features of the optional locking mechanism are described in U.S. Publication No. 2007/0198024 entitled “Curable Material Delivery Device” and the entire teachings of which are incorporated herein by reference. In other embodiments, the handle 46 and/or the handle connector 48 can be omitted.

The access needle assembly 24 is configured to form a channel within bone, and generally includes the access needle 32 that terminates at a distal tip 60. The access needle 32 further includes a distal segment 62 (referenced generally) defining a pre-set memory shape curve or bend 64. As described below, the distal segment 62, and in particular the bend 64, is deflectable, and has a shape memory attribute whereby the distal segment 62 can be forced from the curved shape (shown in FIG. 1) toward a more straightened shape, and will naturally revert back to or toward the pre-set curved shape upon removal of the force. An outer diameter of at least the distal segment 62 is slightly less than a diameter of the cannula lumen 42 (FIG. 5A) such that the distal segment 62 is configured to be slidably received within the guide cannula 30.

The access needle 32 defines a continuous length between a proximal end 66 and the distal tip 60, with the deflectable distal segment 62, and in particular the bend 64, extending along approximately 10%-50% of a length of the access needle 32 as measured from the distal tip 60. To facilitate formation of a curved channel within a confined bone site, the deflectable distal segment 62 can be formed to define the bend 64 at a predetermined radius of curvature appropriate for the procedure in question. In one construction, the bend 64 is J-shaped (approximating at least a 90° bend relative to a central axis of the needle 32; alternatively approximating at least a 120° bend). Alternatively, the bend angle can be greater or lesser depending upon the particular procedure for which the access needle 32 is to be employed.

To facilitate ready deflection of the deflectable distal segment 62 from the curved shape toward a more straightened shape (such as when the distal segment 62 is inserted within the guide cannula 30) and self-reversion back toward the curved shape, the access needle 32, or at least the deflectable curved distal segment 62, is formed of a shape memory material. In some constructions, the access needle 32, or at least the distal segment 62, comprises Nitinol™, a known shape memory alloy of nickel and titanium. For example, the bend 64 can be formed on the distal segment 62 by deforming a straight tube or wire under extreme heat for a prescribed period of time, which pre-sets a curved shape in the distal segment 62. Alternatively, the pre-set bend 64 can be formed in an initially straight tube or wire by cold working the straight shaft and applying a mechanical stress. Cold working permanently locks a crystalline structure (for example, a partial martenisitic crystalline structure) in a portion (i.e., the deflectable distal segment 62) of the shaft, while an unstressed portion remains in, for example, an austenitic structure.

In addition to Nitinol™, other materials exhibiting the above-described shape memory behavior can be employed, including super elastic or pseudoelastic copper alloys, such as alloys of copper, aluminum, and nickel, and alloys of copper, aluminum, and zinc, and alloys of copper and zinc. The deflectable distal segment 62 is formed to be resilient, and to naturally assume the pre-set radius of curvature. In this manner, after the distal segment 62 has flexed or deflected to a substantially straightened shape (not shown), upon subsequent relaxation, the deflectable distal segment 62 “remembers” the pre-set curved shape and relaxes/returns to form the bend 64 as described in greater detail below. In yet other embodiments, the curved shape of the distal segment 62 can be effectuated by one or more additional bodies or mechanisms, such as an internal pull wire. Regardless, the access needle 30, including the distal segment 62, is longitudinally rigid, such that a distal pushing force applied at or adjacent the proximal end 66 is transferred to the distal tip 60. The longitudinal rigidity of the needle 32 is such that when the distal tip 60 is in contact with cancellous bone and the applied pushing force is sufficient for the distal tip 60 to bore through cancellous bone, the needle 32 will not longitudinally buckle or collapse.

In some embodiments, one or more side orifices 70 can be provided adjacent the distal tip 60, extending through a thickness of a side wall of the needle 32. In one construction, a single orifice 70 is provided, and is located “opposite” a direction of the bend 64. In other words, relative to the longitudinal view of FIG. 1, a direction of the bend 64 serves to define an interior bend side 72 and an exterior bend side 74 along the access needle 32. With these designations in mind, the side orifice 70, where provided, is optionally disposed along the exterior bend side 74. Material (e.g., curable material) can be dispensed from the orifice(s) 70, and/or material (e.g., intervertebral disc nucleus) can be aspirated into the orifice(s) 70. In other embodiments, the orifice 70 can be formed at the distal tip 60.

The distal tip 60 can assume various forms configured to effectuate boring through bone (and in particular cancellous bone). As described below, the access needle 32 effectuates formation of a channel in cancellous bone by forcibly advancing the distal tip 60 through the bone material. With this technique, the needle 32 is not rotated or otherwise operated to mechanically cut the bone tissue; instead, the forced advancement of the distal tip 60 compacts and/or crushes bone material in contact therewith to thereby create a space or channel. Thus, the distal tip 60 can have various shapes or tapers appropriate for boring through cancellous bone when forcibly advanced through the cancellous bone. For example, the distal tip 60 can have a bevel at one side thereof, three or more bevel faces, etc.

The access needle assembly 24 can optionally include other components, such as a handle 80 attached to the proximal end 66 of the needle 32. Where provided, the handle 80 facilitates application of a pushing force onto the needle 32. Where provided, the distal end 66 extends within the handle 80, such that a lumen of the needle 32 is open or otherwise accessible through the handle 80. Further, the handle 80 can include indicia 82 that visually indicates a direction of the bend 64, and the handle 80 can be adapted to interface with the optional handle connector 48 of the access needle assembly 22. In other embodiments, the handle 80 is omitted.

The optional material delivery device 34 includes a source 90 of curable material that can assume any form appropriate for delivering the desired curable material. Typically, the source 90 of curable material includes a chamber filled with a volume of curable material and employing any suitable injection system or pumping mechanism to transmit curable material out of the chamber. For example, a hand injection system can be used where a user applies a force by hand to an injector. The force is translated into pressure on the curable material, forcing the curable material to flow out of the chamber. A motorized system may also be used to apply a force.

Tubing 92 is fluidly connected to, and extends from, the source 90 of curable material, and serves as a conduit through which the curable material is delivered. In some embodiments the tubing 92 is configured for connection to the access needle assembly 24, with the access needle 32, in turn, being employed to deliver the curable material to the delivery site. In other embodiments, the tubing 92 can be directed through the guide cannula 30 to deliver the curable material directly to the delivery site.

Where provided, the optional cavity forming device 36 can assume various forms appropriate for forming a void or cavity within bone, and generally includes an elongated body 110 distally connected to or forming a working end 112. The elongated body 110 is sized to be inserted within the cannula lumen 42 (FIG. 5A), and can include one or more tubes, shafts, etc., necessary for operation of the working end 112.

A proximal region 114 of the elongated body 110 is optionally connected to or forms a connector 116. The connector 116 can assume various forms, such as the Y-type connector shown that provides ports fluidly open to various lumen(s) of the elongated body 110 to facilitate operation of the working end 112. Optionally, the connector 116 can include or form features conducive to selective, rigid attachment to the handle connector 48 as described above (e.g., the connector 116 and the handle connector 48 collectively forming a locking mechanism). In other embodiments, the connector 116 is omitted.

The working end 112 can include one or more components adapted for forming a cavity or void within bone. For example, in some constructions, the working end 112 includes one or more expandable or inflatable members (e.g., a single balloon, multiple balloons, a single balloon with two or more discernable inflation zones, etc.), constructed to transition between a contracted (e.g., deflated) state in which the working end/balloon 112 can be passed through the guide cannula lumen 42 (FIG. 5A), and an expanded (e.g., inflated) state in which the working end/balloon end 112 expands and compacts compacted cancellous bone. In this regard, a size and shape of the working end/balloon 112 can be predetermined and/or restrained with one or more additional components (not shown), such as internal or external restraints. Regardless, the working end/balloon 112 is structurally robust, able to withstand (e.g., not burst) expected inflation pressures when in contact with cancellous bone.

The cavity forming device 36 can include one or more additional components connected to or operable with the proximal region 114 for actuating the working end 112. By way of one non-limiting example, then, the cavity forming device 36 can include a source (not shown) of pressurized fluid (e.g., contrast medium) for inflating the balloon(s) carried or formed by the working end 112. A hand-held syringe-type pump can be used as a pressurized source.

With constructions of the cavity forming device 36 incorporating a balloon(s) as the working end 112, at least a distal region 118 (including the working end/balloon 112 of the elongated body 110) is relatively flexible, and readily conforms to different shapes (in longitudinal extension) in response to external forces. Thus, while FIG. 1 illustrates the distal region 118 as being relatively linear in longitudinal extension, the distal region 118 will conform to multiple other shapes, such as the shape of a curved channel formed in cancellous bone as described in greater detail below. For example, the elongated body 110 can be a catheter-type, flexible tube forming one (or more) ports that are fluidly open to an interior of the balloon 112. With these embodiments, the catheter body 110 exhibits sufficient longitudinal rigidity to facilitate distal movement of the balloon 112 through a channel, with the distal region 118 following or conforming to a path of the channel.

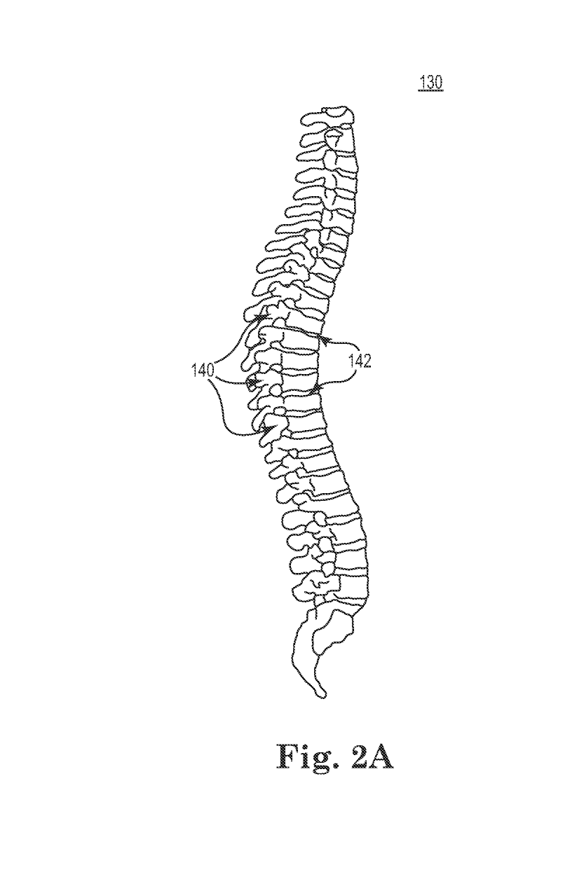

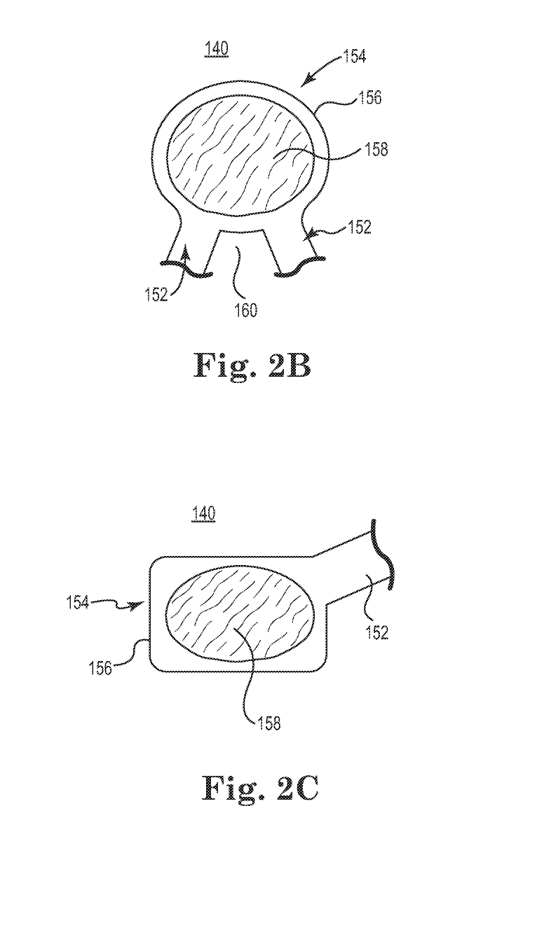

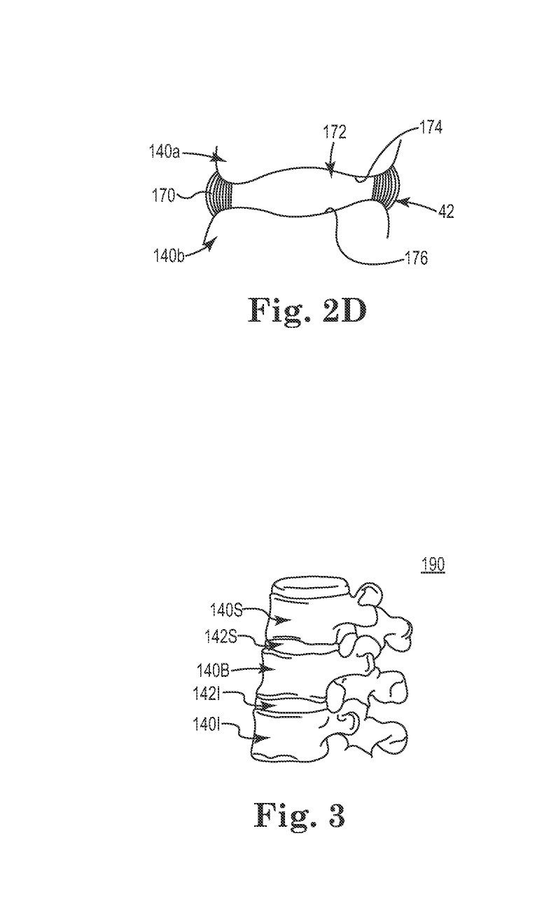

Regardless of an exact configuration, systems 20 in accordance with principles of the present disclosure are useful in performing various treatments on a patient's spine. As shown in FIG. 2A, a patient's spine 130 consists of a number of vertebrae 140, adjacent ones of which are connected by an intervertebral disc 142. FIGS. 2B and 2C are simplified views showing one of the vertebra 140 in greater detail. In general terms, the vertebra 140 includes pedicles 152 and a vertebral body 154 defining a vertebral wall 156 surrounding bodily material 158 (e.g., cancellous bone, blood, marrow, and soft tissue). The pedicles 152 extend from the vertebral body 154 and surround a vertebral foramen 160. With additional reference to FIG. 2D, the intervertebral disc 142 includes or is formed by an annulus 170 and a nucleus 172. The annulus 170 is connected to, and extends between, the opposing vertebrae (140a, 140b in FIG. 2D), and contains the nucleus 172. The nucleus 172 is further bounded or contained by end plates 174, 176 formed by a corresponding surface of the opposing vertebrae 140a, 140b. In other words, in the view of FIG. 2D, the first end plate 174 is formed by the superior vertebra 140a and the second end plate 176 is formed by the inferior vertebra 140b.

With the anatomy of the spine 130 in mind, some methods in accordance with principles of the present disclosure entail accessing and treating one or more levels of the spine 130 through a single insertion path/skin piercing formed to one of the vertebrae (with the vertebra at which the single insertion path is formed being referenced below as a “base” vertebra for ease of explanation). For example, in the view of FIG. 3, treatments can be formed on a spinal segment 190 consisting of a base vertebra 140B, immediately adjacent superior and inferior vertebrae 140S, 140I, and superior and inferior intervertebral discs 142S, 142I. The superior intervertebral disc 142S connects the base vertebra 140B and the superior vertebra 140S, whereas the inferior intervertebral disc 142I connects the base vertebra 140B and the inferior vertebra 140I.

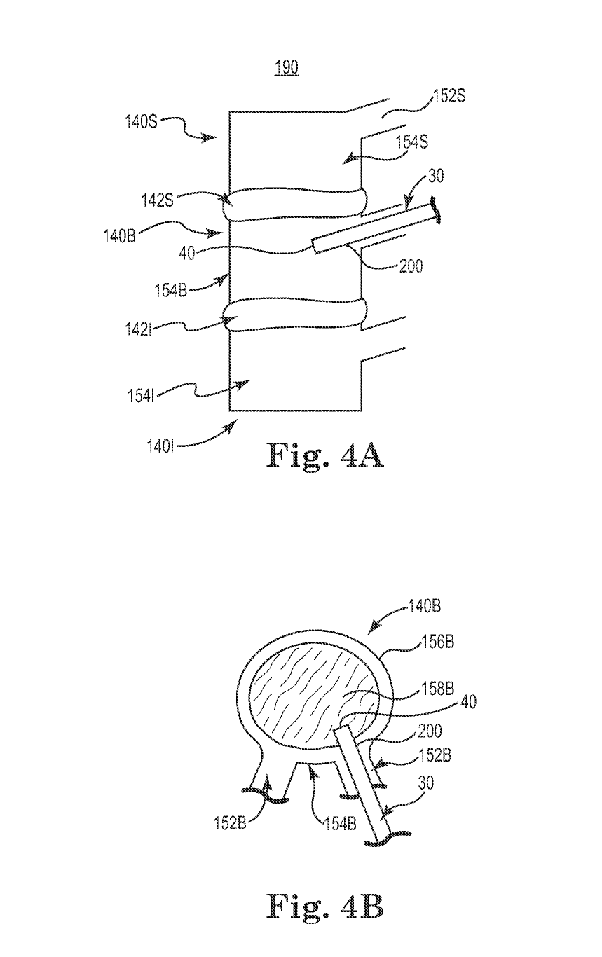

With reference to FIGS. 4A and 4B, some methods in accordance with principles of the present disclosure entail the guide cannula 30 being initially employed to pierce the patient's skin and define an insertion path 200 (referenced generally) into the base vertebral body 154B. In this regard, the insertion path 200 can be formed through one of the base vertebra pedicles 152B and into the bodily material 158B. Thus, as illustrated, the guide cannula 30 has been driven through the base vertebra pedicle 152B via a transpedicular approach. The transpedicular approach locates the cannula 30 between the transverse process and mammilary process of the base vertebra 140B. Alternatively, other approaches into the base vertebral body 154B can be employed (e.g., an anterior or parapedicular approach). In any event, the so-located guide cannula 30 provides general access to an interior of the base vertebral body 154B at the open, distal end 40. In some embodiments, a stylet (not shown) can be employed to assist in forming the insertion access point or path 200 into the base vertebral body 154B.

With reference to FIGS. 5A and 5B, the access needle 32 is deployed through the guide cannula 30 to create a curved channel 202 (referenced generally in FIG. 5B) in the cancellous bone (or other bodily material 158B of the base vertebral body 154B). In particular, the distal segment 62 of the access needle 32 is slidably inserted/distally advanced within the cannula 30. In FIG. 5A, the distal tip 60 of the access needle 32 is poised at the distal end 40 of the cannula 30. Prior to further distal movement, the distal segment 62 is entirely within the cannula lumen 42, such that the distal segment 62 is constrained (e.g., deflected or flexed) to a more straightened shape that generally conforms to a shape of the cannula 32 The force is effectively imparted by the cannula 30 onto the deflectable distal segment 62 due to the radius of curvature defined by the distal segment 62 in a “natural” state being larger than a diameter of the cannula lumen 42. This interaction essentially “removes” the pre-set curvature of the bend 64 (FIG. 1) forcing or rendering the deflectable distal segment 62 to a more straightened state. It will be understood that because an inner diameter of the cannula 30 is greater than a diameter of the access needle 32, the distal segment 62 may continue to have a slight curvature within the cannula 30. Thus, “substantially straightened” is in reference to the access needle 32 being substantially, but not necessarily entirely, linear. Prior to interaction with the cancellous bone material 158B, then, the access needle 32 is flexed toward a substantially or more straightened state within the guide cannula 30.

The access needle 32, and in particular the distal segment 62, is then distally advanced relative to the guide cannula 30 such that at least a portion of the distal segment 62 extends beyond the open distal end 40 of the cannula 30 and into the base vertebra cancellous bone 158B as shown in FIG. 5B. The now unrestrained portion of the distal segment 62 naturally deflects laterally (from the more straightened shape described above) upon exiting the cannula distal end 40, self-reverting to or toward the pre-set curvature of the bend 64 previously described due to, for example, the shape memory characteristic. In addition, with distal advancement of the distal segment 62 from the cannula 30, the distal tip 60 intimately contacts and effectively compacts or crushes the base vertebra cancellous bone 158B. Stated otherwise, the area of cancellous bone 158B directly contacted by the advancing distal tip 60 is permanently deformed or compacted, resulting in formation of the channel 202. Taken in combination, then, the channel forming effects of the distal tip 60 and the pre-set curved shape of the distal segment 62 produces or generates the curved channel 202 in response to a distally-directed pushing force applied to the proximal end 66 (FIG. 1) of the access needle 32 in a direction generally co-axial with the central axis C of the guide cannula 30 as shown in FIG. 5B. The pushing force is translated to the distal tip 60, and is of sufficient magnitude to cause compaction or crushing of the contacted cancellous bone 158B. Further, the self-reverting curved shape of the distal segment 62 effectively “directs” the distal tip 60 through a curved or arcuate path while boring through the cancellous bone 158B.

As reflected in FIG. 5B, spatial arrangement of the access needle 32, and in particular the bend 64 in the distal segment 62, relative to the spinal segment 190 is such that the arcuate path 202 formed by distal advancement of the access needle 32 proceeds or is “aimed” toward the superior vertebral body 154S. That is to say, with the methodology implicated by FIG. 5B, the clinician intends to perform a treatment on the superior vertebral body 154S, and thus rotationally arranges the access needle 32 relative to the spinal segment 190 such that the distally-advancing distal tip 60 proceeds (via the self-reverting nature of the distal segment 62 back toward the curved shape) toward the superior vertebral body 154S. As shown in FIG. 5C, with further distal advancement of the access needle 32, the distal tip 60 passes through the superior intervertebral disc 142S (including the second end plate 176S, the nucleus 172S, and the first end plate 174S) and into the superior vertebral body 154S. Advancement of the access needle 32 continues until the distal tip 60 is located at, or approximately at, a target site 204 within the superior vertebral body 154S. Notably, the access needle 32 creates the curved channel 202 independent of any naturally occurring “paths” within the cancellous bone 158B. For example, the natural anatomy of the cancellous bone and/or occurring debris within the vertebral bodies 154B, 154S may tend to inherently direct an otherwise flexible tube (with no pre-set longitudinal curve) toward or away from the target site 204, somewhat like a grain pattern in wood. Under either circumstance, the access needle 32 and corresponding methods of use of the present disclosure definitively achieve the curved channel 202 as a direct function of the pre-set curve in the access needle 32. Thus, the present disclosure is distinct from a non-linear channel formed by a flexible tube that simply happens to deflect when encountering the natural anatomy.

A vertebroplasty procedure can then be performed on the superior vertebral body 154S as shown in FIG. 5D. For example, curable material 210 is delivered to the target site 204 in the superior vertebral body 154S via the access needle 32. Alternatively, the access needle 32 can be removed from the guide cannula 30, and replaced by a separate tubing (not shown) that is otherwise directed to the target site 204 via the previously-formed curved path 202.

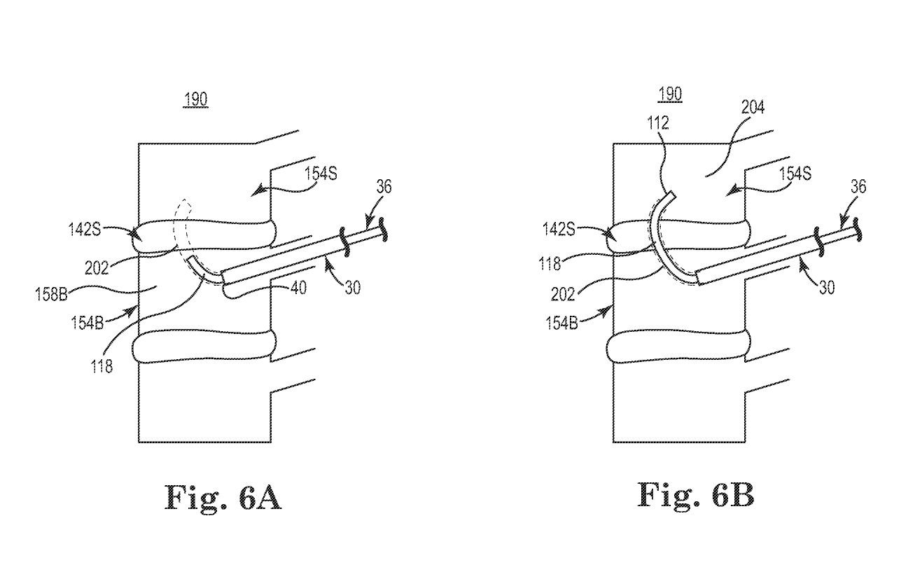

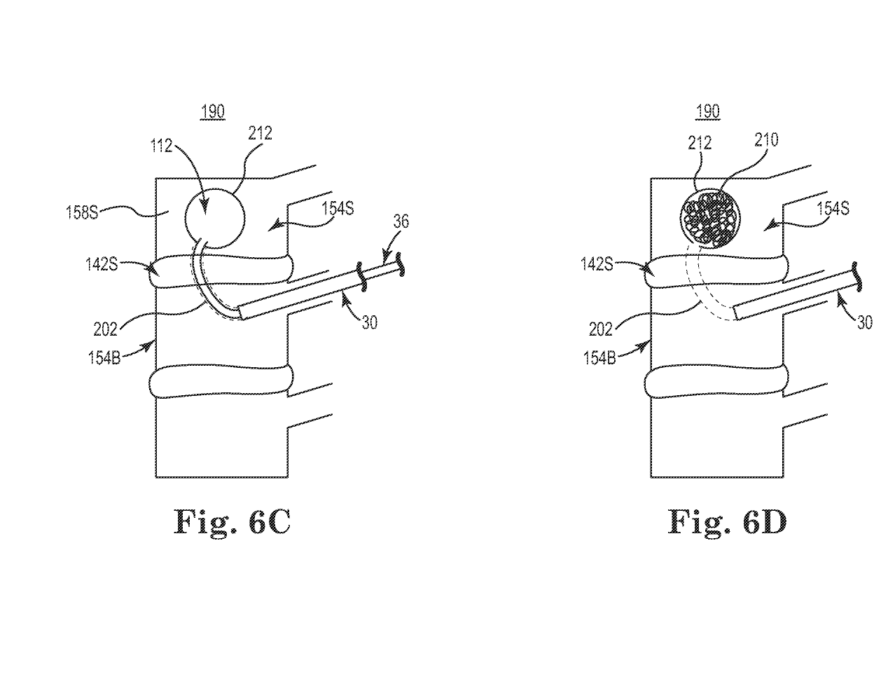

In yet other embodiments, after accessing the target site 204 with the access needle 32 but prior to delivering the curable material 210, the access needle 32 is removed and is replaced by the cavity forming device 36 as shown in FIG. 6A. In particular, the distal region 118 is inserted through, and is distally advanced from, the guide cannula 30. In this regard, as portions of the distal region 118 exit the cannula distal end 40, the distal region 118 follows a path of the curved channel 202. More particularly, the distal region 118 is sufficiently flexible such that upon contacting a wall of the curved channel 202 and with further distal advancement, the distal region 118 readily deflects, thereby tracking or following the shape of the curved channel 202. The distal region 118 follows the path of least resistance and does not bore through the cancellous bone 158B surrounding the curved channel 202. Distal advancement of the distal region 118 continues through the curved channel 202, resulting in the arrangement of FIG. 6B. In the final location, the working end 112 is at or immediately proximate the target site 204.

With reference to FIG. 6C, the cavity forming device 36 is then operated to cause the working end/balloon 112 to form a cavity or void 212 (referenced generally) in the cancellous bone (or other bodily material 158S of the superior vertebral body 154S). For example, the working end/balloon 112 can be expanded (e.g., inflated). The working end/balloon 112 is then transitioned to the contracted state (e.g., deflated), and removed from the guide cannula 30. As shown in FIG. 6D, the resultant cavity 212 is then filled with the curable material 210, for example via the access needle 32 (FIG. 1) as described above.

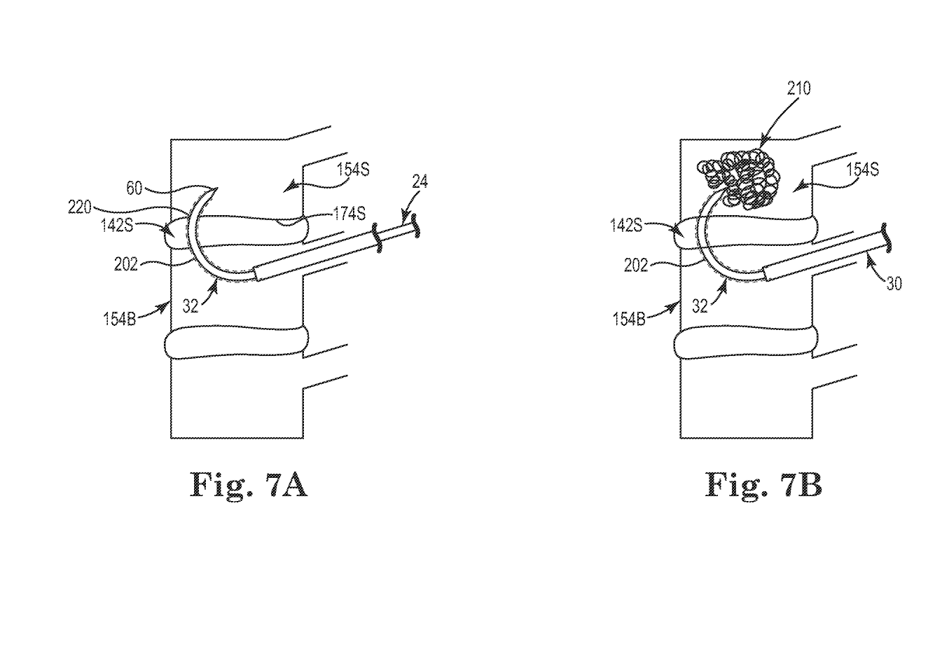

Regardless of whether the cavity 212 is formed, yet other methods in accordance with principles of the present disclosure entail obstructing a portion of the curved channel 202 immediately prior to delivery of the curable material 210. For example, as shown in FIG. 7A, following location of the distal tip 60 within the superior vertebral body 154S, an obstruction body 220 is deployed across the curved channel 202 in region of the end plate 174S formed by the superior vertebral body 154S (and otherwise associated with the superior intervertebral disc 142S). The obstruction body 220 can assume various forms, and can be deployed in various manners. In some embodiments, the access needle assembly 24 (referenced generally) provides the access needle 32 as dual lumen needle, with the obstruction body 220 being a balloon carried by the needle 32 and fluidly connected to one of the lumens. With this construction, the access needle assembly 24 is operated to inflate the obstructing body/balloon 220, effectively sealing the channel 202 proximate the end plate 174S. Subsequently, and as shown in FIG. 7B, the delivered curable material 210 is prevented from undesirably flowing through the channel 202 and into the superior intervertebral disc 142S. The obstruction body 220 can have other constructions, and may or may not be carried by the access creating needle 32. Alternatively, the obstruction body 220 and related methods of use can be omitted.

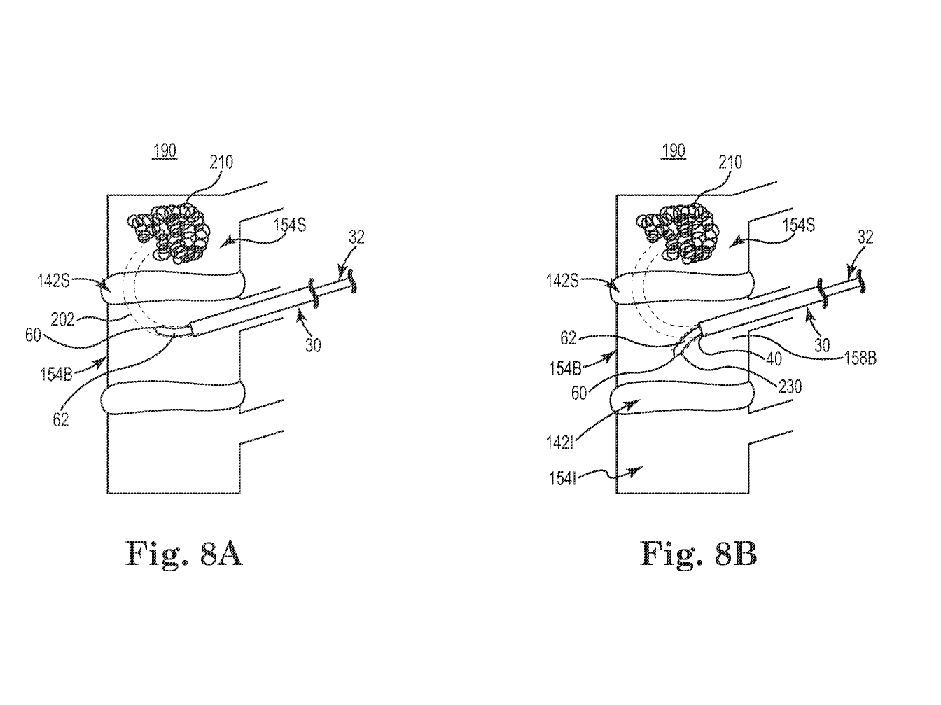

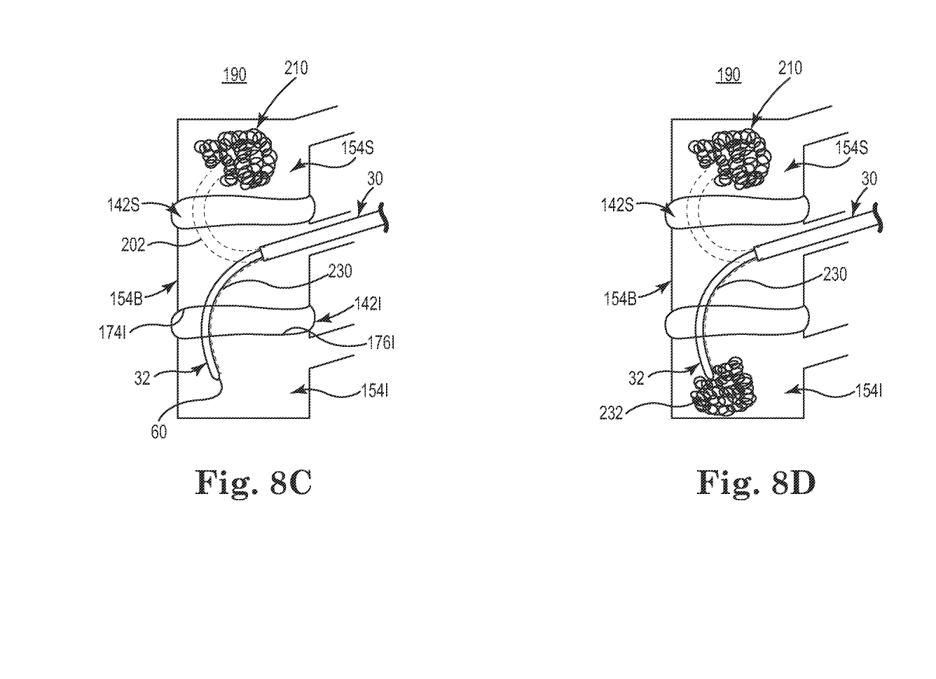

Following delivery of the curable material 210 into the superior vertebral body 154S, methods of the present disclosure include performing vertebroplasty on other regions of the spinal segment 190. For example, with reference to FIG. 8A, the access needle 32 is proximally retracted relative to the guide cannula 30, withdrawing the distal segment 62 and the distal tip 60 back within the cannula lumen 42 (FIG. 5A). As described above, once inside the cannula 30, the distal segment 62 is forced to the more straightened shape. The access needle 32 is then rotated relative to the guide cannula 30, for example approximately 180°. The access needle 32 is then distally advanced relative to the cannula 30, forcing the distal tip 60 and the distal segment 62 distally beyond the cannula distal end 40 as shown in FIG. 8B. Distal advancement of the access needle 32 creates a second curved channel 230 within the base vertebral body 154B. The curved shape of the second channel 230 is again dictated by the self-achieving curved format of the distal segment 62 as the distal tip 60 is forced through the cancellous bone 158B of the base vertebral body 154B. With reference to FIG. 8C, distal advancement of the access needle 32 continues, with the distal tip 60 being forced through the inferior intervertebral disc 142I (including the end plates 174I, 176I at opposite sides of the inferior disc 142I), and into the inferior vertebral body 154I. Once desirably located, curable material 232 is delivered into the inferior vertebral body 154I in accordance with previous descriptions and as reflected by FIG. 8D. Once again, a cavity can be formed in the inferior vertebral body 154I and/or the second curved channel 230 obstructed prior to delivery of the curable material 232.

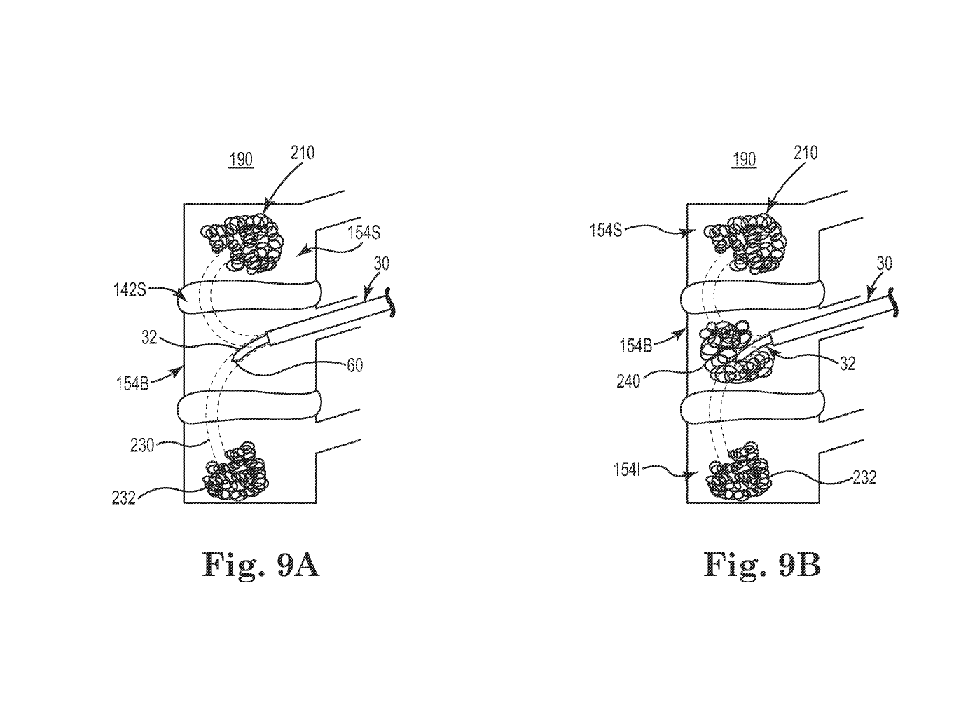

In related methods, and as shown in FIG. 9A, the access needle 32 can again be distally retracted relative to the guide cannula 30, locating the distal tip 60 within the base vertebral body 154B. Vertebroplasty can then performed on the base vertebral body 154B by delivering curable material 240 thereto as shown in FIG. 9B. The guide cannula 30 and the access needle 32 are then removed from the patient.

Based upon the above descriptions, methods in accordance with principles of the present disclosure can beneficially provide vertebroplasty treatments to three vertebral bodies via a single access point/skin piercing. In related embodiments, methods of the present disclosure include delivery of curable material into only two of the vertebral bodies 154B, 154S, or 154I.

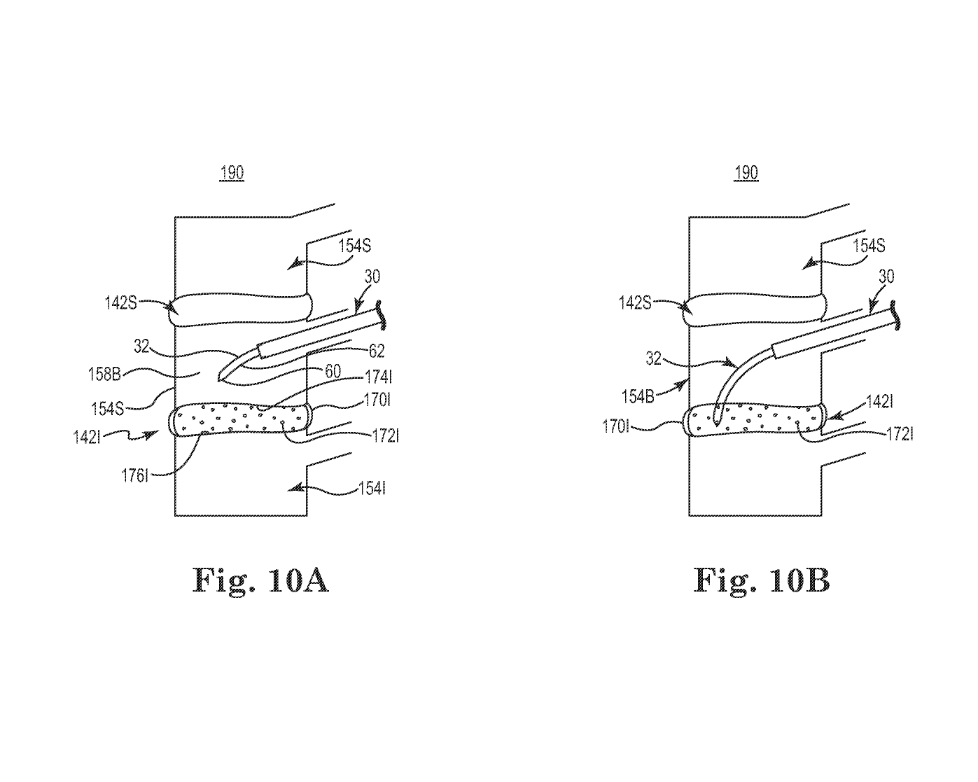

Another spinal treatment procedure in accordance with methods of the present disclosure is shown in FIGS. 10A and 10B. In FIG. 10A, the distal end 40 of the guide cannula 30 is lodged within the base vertebral body 154B as described above (e.g., percutaneously via a pedicular approach). The access needle 32 is distally advanced relative to the cannula 30, forcing the distal tip 60 through the cancellous bone 158B of the base vertebral body 154B, through the first end plate 174I at the inferior intervertebral disc 142I, and into the nucleus material 172I. Once again, the self-reverting memory set shape characteristic of the distal segment 62 causes the distal tip 60 to create and follow a curved path, and thus is naturally directed or “aimed” from the base vertebral body 154B into the inferior intervertebral disc 142I. With reference to FIG. 10B, with the distal tip 60 now within the nucleus 172I, material of the nucleus 172I can be removed, for example aspirated through the access needle 32. Thus, methods of the present disclosure entail a disc decompression procedure via an access point apart from the otherwise frangible annulus 170I. As a point of reference, disc decompression is conventionally performed when a patient has a bulging disc that presses on nerves, causing pain. Removing some of the material of the nucleus 172I relieves some of the pressure on the disc/nerves, and the bulge can recede, eliminating the pain. Accessing the nucleus 172I via the adjacent vertebral body 154B can be beneficial because it eliminates the need to navigate around the multitude of nerves to properly position the needle distal tip 60. If desired, the access needle 32 can subsequently be reoriented relative to the guide cannula 30 and the spinal segment 190, and then distally advanced to locate the distal tip 60 within the superior intervertebral disc 142S for an additional decompression procedure.

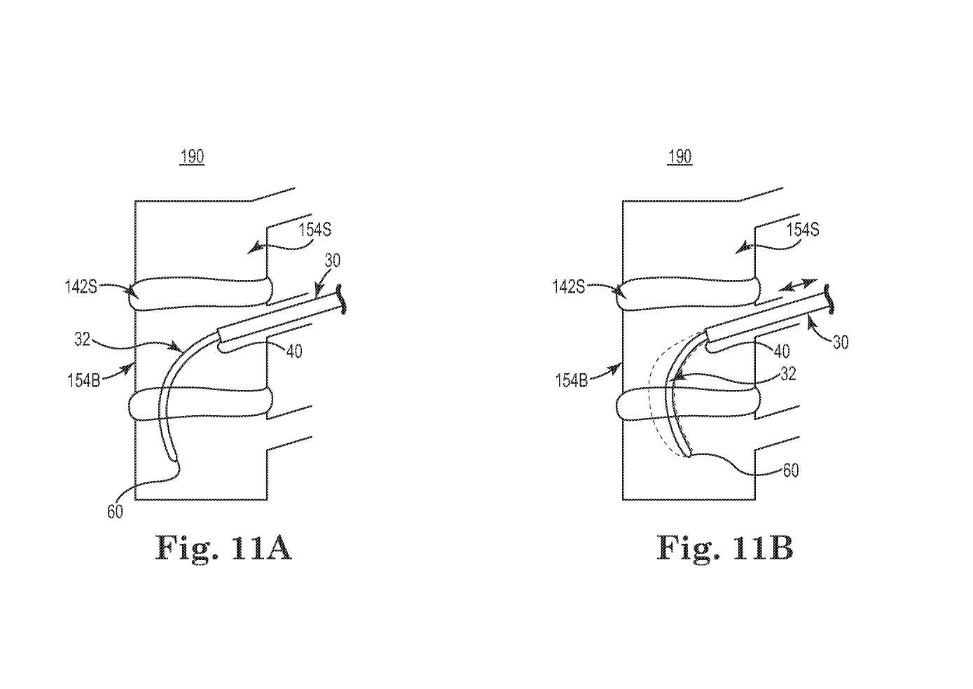

With any of the methodologies described above, desired location of the access needle's distal tip 60 relative to the vertebral body or intervertebral disc in question can be achieved by adjusting or manipulating a location of the guide cannula distal end 40 relative to the base vertebral body 154B as the access needle 32 is being distally advanced. As a point of reference, FIGS. 11A and 11B illustrate a comparison of the paths of travel of the distal tip 60 where the cannula 30 remains stationary (FIG. 11A) and where the cannula 30 is spatially manipulated during advancement of the access needle 32 (FIG. 11B).

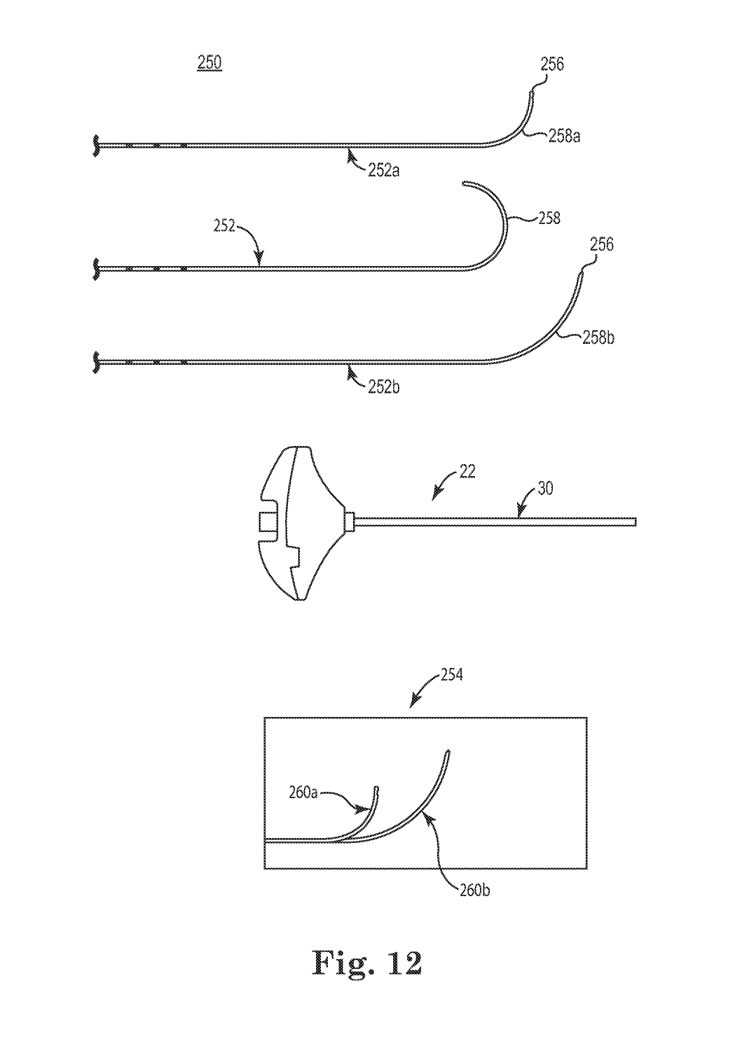

To better accommodate the anatomy of a particular patient, other embodiments of the present disclosure provide a kit 250 for treating a patient's spine as shown in FIG. 12. The kit 250 includes the cannula assembly 22 (including the guide cannula 30) as described above, along with a plurality of access needles 252 and a template 254. The access needles 252 are highly akin to the access needle 32 (FIG. 1) described above, each terminating at a distal tip 256 and forming a memory shape curved distal segment 258. However, at least two of the access needles 252 (e.g., the needles 252a, 252b identified in FIG. 12) differ from one another in terms of a radius of curvature of the corresponding distal segment 258 and/or a length of the corresponding distal segment 258. For example, the distal segment 258 of the first access needle 252a is shorter and has a smaller radius of curvature as compared to the second access needle 252b.

The template 254 provides a visual representation or display of the difference(s) between the distal segments 258 of the access needles 252. For example, the template 254 can include or display indicia (e.g., pictures or drawings) of the distal segments 258 of each of the access needles 252 provided with the kit 250. Thus, FIG. 12 reflects the template 254 as including a representation 260a of the distal segment 258a of the first access needle 252a, and a representation 260b of the distal segment 258b of the second access needle 252b. Alternatively, the template 254 can utilize other conventions or nomenclatures to visually indicate differences between the access needles 252 provided with the kit 250.

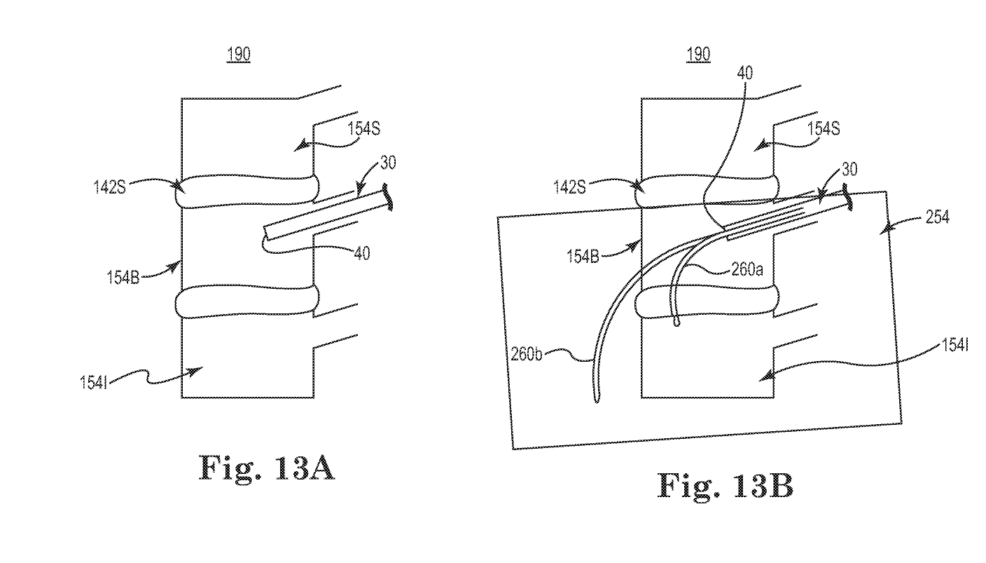

During use of the kit 250 in treating a patient's spine, the guide cannula 30 is lodged within the base vertebral body 154B as described above and as shown in FIG. 13A. Once lodged, an image of the spinal segment 190 is obtained (e.g., x-ray). The view of FIG. 13A is indicative of a so-obtained image. With reference to FIG. 13B, the template 254 is then correlated with the spinal segment image, aligning the curved channel representations provided on the template 254 with the distal end 40 of the cannula 30 in the spinal segment image.

The various access needle channel representations on the template 254 are then compared with the spinal segment image to determine which curved path reflected in the template 254 best meets the anatomical constraints of the spinal segment 190 for locating a distal tip of a subsequently-deployed access needle within the desired region of the spinal segment. For example, with procedures in which the clinician desires to access the inferior vertebral body 154I (for subsequent delivery of curable material therein), the clinician reviews the spinal segment image/template 254 arrangement of FIG. 13B, and can visually determine that the first needle representation 260a readily proceeds within the base vertebral body 154B and into the inferior vertebral body 154I, whereas the second needle representation 260b does not. Based upon this review, the clinician is advised to select the first access needle 252a (FIG. 13A) to access and treat the inferior vertebral body 154I as described above. Where access to and treatment of the superior vertebral body 154S is desired, a similar comparison of the template 254 and the spinal segment image will reveal which of the available access needles 252 (FIG. 13A) is best suited for deployment within the spinal segment 190. Thus, with the kits 250 of the present disclosure, corresponding methods of use can entail deployment of a first one of the available access needles 252 to access and treat a first region of the spinal segment, and use of a second, different one of the available access needles 252 to access and treat another region of the spinal segment 190.

The systems, kits, and methods of the present disclosure provide a marked improvement over previous designs. Vertebroplasty or other spinal treatment procedures can be performed at multiple spinal segment regions via a single needle puncture or access point. In other embodiments, a flexible drill can be used in place of the access needle 32. Flexible drills for vertebral augmentation, for example available from Soteira, Inc. (Natick, Mass.) and Osseon, Therapuetics (Santa Rosa, Calif. under the tradename Osseoflex DR Steerable Bone Drill), generally include a flexible shaft that has either a shape memory curvature or incorporates other features permitting a user to effectuate as desired bend. The flexible drill can be used with any of the methodologies described above.

Although the present disclosure has been described with reference to preferred embodiments, workers skilled in the art will recognize that changes can be made in form and detail without departing from the spirit and scope of the present disclosure.

1. A method for treating a spine of a patient, the method comprising:
inserting a distal end of a guide cannula into a first vertebral body of the spine, the guide cannula defining a linear lumen, and wherein:
the first vertebral body is connected to a second, immediately adjacent vertebral body by a first disc, the first disc including an annulus and a nucleus, and bounded by a first end plate formed by the first vertebral body and a second end plate formed by the second vertebral body;
inserting a distal segment of an access needle into the guide cannula lumen, wherein the distal segment terminates at a distal tip and has a shape memory characteristic naturally assuming a curved shape in longitudinal extension, and further wherein the guide cannula forces the distal section to deflect from the curved shape toward a straightened shape when the distal segment is within the lumen;
distally advancing the distal segment from the distal end and into bone structure of the first vertebral body, wherein at least a portion of the access needle distal segment distal the guide cannula distal end naturally reverts toward the curved shape;
further distally advancing the access needle relative to the guide cannula, including the distal tip progressing through the bone structure and the first end plate of the first vertebral body; and
altering a structure of the spine by at least one of:
delivering a curable material into the second vertebral body through the access needle,
operating a cavity forming device delivered through a channel created by the access needle to form a cavity in the second vertebral body, and
removing a portion of the nucleus through the access needle.

2. The method of claim 1, wherein the step of inserting a distal end of the guide cannula includes guiding the distal end into the first vertebral body via a posterior pedicular approach.

3. The method of claim 1, wherein the step of further distally advancing the access needle includes the distal tip generating a curved channel in the bone structure of the first vertebral body, the curved channel defining a curve relative to a central axis of the guide cannula lumen.

4. The method of claim 1, further comprising:
receiving a set of available curved needles, each of the available needles including a distal section having a memory set curved shape, wherein a first one of the available needles differs from a second one of the available needles by at least one of a length and radius of curvature of the corresponding distal segment;
following the step of inserting the distal end of the guide cannula into the first vertebral body, evaluating a spatial relationship of the distal end relative to at least one of the first disc and the second vertebral body;
and
selecting one of the first and second available curved needles based upon the evaluation;
wherein the selected available needle is employed as the access needle.

5. The method of claim 4, wherein the step of evaluating includes comparing a representation of the spatial relationship of the distal end relative to one of the first disc and the second vertebral body with a template providing a representation indicative of curvatures of the first and second available curved needles.

6. The method of claim 1, wherein following the step of further distally advancing the access needle, the method further comprising:
even further distally advancing the access needle relative to the guide cannula such that the distal tip pierces through the second end plate and into a bone structure of the second vertebral body;
wherein the step of altering a structure of the spine includes delivering curable material into the second vertebral body.

7. The method of claim 6, wherein prior to the step of delivering curable material into the second vertebral body, the method further comprising:
inflating a balloon to close off a channel created by the access needle at a location of the second end plate.

8. The method of claim 6, wherein following the step of delivering curable material into the second vertebral body, the method further comprising:
proximally retracting the access needle relative to the guide cannula to withdraw the distal tip from the second vertebral body and the first disc, and locate the distal tip within the first vertebral body; and
delivering a curable material into the first vertebral body through the access needle.

9. The method of claim 8, wherein the first vertebral body is inferior to the second vertebral body.

10. The method of claim 6, wherein the spine further includes a third vertebral body immediately adjacent the first vertebral body opposite the second vertebral body, the first and third vertebral bodies connected by a second disc bounded by a first end plate formed by the third vertebral body and a second end plate formed by the first vertebral body, and further wherein following the step of delivering curable material into the second vertebral body, the method further comprising:
proximally retracting the access needle relative to the guide cannula to locate the distal tip within the cannula lumen;
forming a curved channel from the distal end through the second end plate of the first vertebral body, the second disc and into the third vertebral body; and
delivering curable material into the third vertebral body.

11. The method of claim 10, wherein the step of forming a curved channel includes:
rotating the access needle relative to the guide cannula;
distally advancing the access needle relative to the guide cannula such that as the distal tip is advanced distal the cannula distal end and the distal section naturally reverts toward the curved shape, the distal section moving toward the third vertebral body with further distal advancement of the access needle.

12. The method of claim 11, wherein the step of delivering curable material into the third vertebral body occurs through the access needle.

13. The method of claim 12, wherein following the step of delivering curable material into the third vertebral body, the method further comprising:
proximally retracting the access needle relative to the guide cannula to withdraw the distal tip from the third vertebral body and the second disc, and locate the distal tip in the first vertebral body; and
delivering curable material into the first vertebral body through the access needle.

14. The method of claim 1, wherein the step of operating a cavity forming device includes:
removing the access needle from the access needle; and
inserting the cavity forming device through the guide cannula lumen.

15. The method of claim 14, wherein the cavity forming device includes a flexible body carrying a balloon at a distal region thereof, and further wherein operating the cavity forming device includes inflating the balloon within the second vertebral body to form the cavity.

16. The method of claim 15, further comprising:
delivering curable material into the cavity.

17. The method of claim 1, wherein the step of altering a structure of the spine includes aspirating nucleus material through the distal tip.

18. The method of claim 1, wherein the step of further distally advancing the access needle includes:
selectively moving the guide cannula and the access needle relative to one another to alter a geometry of the access needle distal segment distal the guide cannula distal end relative to the first vertebral body.

19. A kit for treating a spine of a patient via a single access point in a vertebral body of the spine, the kit comprising:
a guide cannula having a linear lumen;
a plurality of access needles each sized to be slidably received within the lumen and each including a distal segment terminating at a distal tip, the distal segment having a shape memory characteristic naturally assuming a curved shape in longitudinal extension;
wherein the distal segment of a first one of the access needles differs from the distal segment of a second one of the access needles in terms of at least one of length and radius of curvature.

20. The kit of claim 19, further comprising:
a template visually representing the difference between the first and second access needles.
